(19) United States
(12) Patent Application Publication
(10) Pub. No.: US 2021/0275807 A1
(43) Pub. Date: Sep. 9, 2021

(54) SYSTEM AND METHOD FOR DETERMINING USER INTENTION FROM LIMB OR BODY MOTION OR TRAJECTORY TO CONTROL NEUROMUSCULAR STIMUATION OR PROSTHETIC DEVICE OPERATION

(71) Applicant: Northwell Health, Inc., Manhasset, NY (US)

(72) Inventor: Chad Edward Bouton, Darien, CT (US)

(21) Appl. No.: 17/194,094

(22) Filed: Mar. 5, 2021

Related U.S. Application Data
(60) Provisional application No. 62/985,951, filed on Mar. 6, 2020.

(51) Int. Cl. A61N 1/36; A61N 1/04; A61B 5/11; A61B 5/00; G06N 20/10

(52) U.S. Cl. CPC A61N 1/36003; A61N 1/0452; A61N 1/36062; A61N 1/36103; A61N 1/36053; A61B 2562/0219; A61B 5/1125; A61B 5/4836; G06N 20/10; A61B 2505/09; A61B 5/1122

(57) ABSTRACT

Disclosed is a device for restoring motion to a person's paralyzed body part, for example, the person's hand. The device senses movement of another part of the person's body not affected by the paralysis, for example, the person's arm or shoulder. Motion sensors generate motion signals as the person moves the non-paralyzed body part. A processor stores information associating a predefined trajectory with a particular action, for example, closing the hand to grasp an object. The processor monitors the motion signals and, when the motion corresponds with the predefined trajectory, the processor energizes muscle stimulators connected with muscles that control the paralyzed hand to perform the action, for example, to cause the hand to close around and grasp the object.

This application claims priority under 35 U.S.C. § 119 to U.S. Provisional Patent Application No. 62/985,951, filed on Mar. 6, 2020. The disclosure of that application is incorporated herein by reference.

### BACKGROUND

### Field

This disclosure relates to systems, apparatuses, applications, and methods to assist a partially disabled person by providing volitional movement of a paralyzed joint or prosthetic device by determining the person's intention to move the joint or device from analysis of limb or body movements of the person's able-bodied joints. More particularly, this disclosure relates to a system, method, or device for determining that the general motion (translational and/or rotational motion) or trajectory of a neurologically able limb or other body part is determinative of the user's intention to perform an action using a disabled or missing appendage and, in response to the determined intention, stimulating the neurologically disabled part (via the nerve and/or muscle that controls such part) or a neural target (nerve, spinal cord, or brain) to promote neural growth/regeneration or connection strengthening causing recovery of movement or function, or to control a prosthetic replacement to perform the action. A device according to one embodiment of the disclosure detects the reaching trajectory of a person's arm, discerns the person's intention to grasp an object, and activates or modulates a neuromuscular stimulation device (LAMES) to cause the person's otherwise paralyzed hand (or actuates the person's robotic/prosthetic hand) to open and close to grasp and hold the object.

### Description of the Related Art

Almost 5.4 million people in the United States alone are living with paralysis. Stroke and spinal cord injury are two leading causes. Every year in the U.S. there are more than 17,700 new cases of spinal cord injury (NSCISC, 2019). A majority of these injuries results in incomplete (48%) and complete (20%) quadriplegia, which severely affects arm and hand movements of the survivors and undermines their quality of life.

A top priority for individuals living with quadriplegia is regaining hand function. Various invasive and non-invasive neuromuscular electrical stimulation (LAMES) devices have been proposed to rehabilitate or evoke upper limb and hand movement. These known systems have drawbacks. The Freehand System used shoulder movements coupled to switches that triggered a selected hand motion through electrical muscle stimulation via implanted electrodes. Actuation of switches may be cumbersome and may require the user to perform unnatural motions to operate the muscle stimulator. Such motions may draw attention to the user's disability and may impact how the user is perceived by others. Also, the repertoire of hand motions the user can perform may be limited by the number of switches that can be operated by a user's shoulder muscles.

Other systems may require surgical procedures to implement. For example, some systems rely on to implanted electromyographic sensors to detect a patient's intention to move a disabled or amputated joint. Cortical brain-computer interfaces (BCIs) have been used to control LAMES devices by recording and decoding motor activity in the brain to allow volitional control of an otherwise paralyzed hand. These approaches require implanting electrodes or other structures in the user's body, potentially exposing users to medical risks and adding significant cost.

### SUMMARY

The present disclosure relates to apparatuses and methods to address these difficulties. Patients living with paralysis want to integrate into society without drawing attention to their disability as much as possible. While rehabilitation can restore some patients to at least partial mobility, it may be difficult or impossible to restore fine motor control, for example, to allow a user to reach out and grasp an object like a beverage glass or a piece of food. The present disclosure allows patients suffering from the inability to control grasping motions of their hand to perform tasks such as feeding themselves, without having to resort to tools, such as utensils affixed to their hand, to perform daily activities.

Patients living with paralysis resulting from a stroke, spinal cord injury, or other conditions can lose movement in their hands and/or legs but often can retain residual movement in other areas of their bodies. For example, in a C5 level spinal cord injury, the most common injury level for quadriplegics, movement of the hand is severely impaired, but shoulder movement and elbow flexion are spared. Similarly, after a stroke, gross movement of the arm (shoulder and elbow) can often be regained through intensive rehabilitation but regaining hand movement remains problematic. Finally, a paraplegic or stroke victim may not have use or full use of their legs or may suffer from foot drop (lacking ankle flexion ability), but may have arm movement or trunk or hip movements they can still make.

Disclosed herein are methods and systems that return volitional control of the user's paralyzed joints and/or external devices by sensing and recognizing the movement and trajectories in able-bodied joints the person still possesses. The system discerns the intention of the user to perform an action using the paralyzed or prosthetically replaced joint using computerized algorithms including machine learning that adapt to the user's particular body motions. The detected body motions and trajectories can then be used to drive a wide variety of desired outcomes. According to one embodiment, such a system determines a person's intention to reach out to grasp an object and actuates an LAMES device to open and close the user's paralyzed hand to grasp and hold the object.

The present disclosure includes devices that sense and recognize limb trajectories (e.g., reaching motions controlled by residual shoulder and elbow movements) and other body motions, positions, or orientations to activate muscles of a disabled body part through electrical stimulation via electrodes or electrode arrays, to cause a specific activity, for example, a “key grasp” pinching motion of the hand, and the like, or energize actuators on a prosthetic body part. A variety of predefined trajectories and limb or body motions, which could be a combination of translational and rotational type motions, may be stored, each trajectory or motion associated with a different action. Based on recognized motions, a device according to embodiments of the disclosure can also be used to control of external devices, for example, a computer or motorized wheelchair. Moreover, many distinct trajectories can be identified with different actions, allowing the repertoire of actions available to the user to expand.

The present disclosure also includes devices that recognize motion about able-bodied joints such as the hip, lumbar spine, and knee to identify motions associates with a person's gait and apply stimulation signals to muscles in synchrony with the person's gait. Such a device may be used to restore a more effective gait motion where neurological injury has impaired motion of the person's foot, ankle, or leg. Such a device may be used to strengthen muscles required for walking preoperatively, for example, before a hip or knee replacement procedure, and/or post-operatively as part rehabilitation treatment.

According to another embodiment, instead of, or in addition to energizing electrodes or prosthetic devices to enable movement, a system according to the disclosure delivers electrical stimulation to the site of the neurological injury, or a neural pathway connected to the neurological injury (e.g. spinal cord, brain, or peripheral nerve). By providing electrical stimulation, with electrodes being placed transcutaneously or epidurally, over or near to the site of a spinal cord, nerve, or brain injury, while at the same time moving the affected limb, a system according to the disclosure may assist in repair of injured motor fibers, nerves or neurons. The system may also provide electrical stimulation, with electrodes being placed transcutaneously or epidurally, over or near or superior to the site of the injury, in the case of spinal cord injury, to potentially assist in the healing of damage to sensory fibers, nerves or neurons.

Using sensors on the arms, legs, and/or body, a wide variety of two- and three-dimensional (2D/3D) motions (translational acceleration, rotational velocity, and orientation with respect to earth's magnetic field) can be recognized. According to some embodiments, such motion is detected by inertial motion units (IMUs) that have 3 to 9 degrees-of-freedom in total. According to other embodiments, visual images of motions may be recognized as well. Just as a child traces letters, numbers, and patterns in the air with a sparkler, the device recognizes fluid, natural curvilinear arm reaching trajectories and pre-trained patterns such as well-known script numbers and letters. The user can then perform motions of their choice or natural reaching trajectories, and these motions are recognized and, in turn, used to control various neuromuscular stimulation and prosthetic/robotic devices that facilitate movement in the paralyzed joints. In the arm, movement trajectories of the arm, driven by residual shoulder movements, can be used to drive stimulation or robotic control of multiple wrist, hand, and finger movements (or external devices such as a computer, stereo, etc.).

In addition to enabling patients to grasp objects using residual mobility, a device according to embodiments of the disclosure may improve neurological function by providing feedback to the patient's central nervous system to associate motions of able joints and limbs with activation of the disabled body part. Thus, using such a device to drive neuromuscular or robotic-driven movement in paralyzed joints, has assistive, rehabilitative, and therapeutic applications in stroke, spinal cord injury, and other neurodegenerative conditions. This approach also has application in general physical therapy after injury or surgery to the hand, foot, leg, or other parts of the body.

Furthermore, the disclosed embodiments can be used to measure, track, and recognize (through machine learning algorithms such as those disclosed) the quality of limb/body movement trajectories over time in rehabilitative applications. Because motion of joints is captured, recorded, and recognized or graded, a physical therapist can monitor a patient's progress and tailor the therapy to address particular parts of body motion that may be problematic. Machine learning or other forms of artificial intelligence, including deep learning methods, can be used to analyze aggregate data (from many anonymous patients) to find general patterns and metrics indicating progress or setbacks and issues that can be flagged for review or corrective action.

According to one embodiment a device is disclosed comprising one or more motion sensors, the sensors generating one or more respective motion signals indicative of movement of a first body part of a human, a muscle stimulator, wherein the muscle stimulator generates one or more stimulation signals to cause one or more muscles to displace a second body part to perform at least one action, and a processor connected with the one or more motion sensors and the muscle stimulator. The processor includes data storage, the data storage including at least one expected trajectory associated with an intention of the human to perform the at least one action. The processor receives the one or more signals from the one or more motion sensors, calculates an actual trajectory of the first body part, compares the actual trajectory with the expected trajectory, and, based on the comparison, actuates the muscle stimulator to displace the second body part to perform the at least one action. The processor may compute a difference between the actual trajectory and the expected trajectory and perform the comparison and actuate the muscle stimulator based on the difference.

According to one embodiment the at least one action comprises a plurality of actions and the at least one expected trajectory comprises a plurality of expected trajectories. Each of the plurality of expected trajectories is associated with at least one of the plurality of actions. The processor compares the actual trajectory with the plurality of expected trajectories to identify a first trajectory associated with a first action of the plurality of actions, and processor actuates the muscle stimulator to perform the first action. The device may comprise an input device connected with the processor where the input device adapted to a receive a feedback signal. The feedback signal may indicate that the action was the intended action of the human. The processor may generate the expected trajectory based on a training set of motions. The one or more stimulation signals to perform the at least one action may comprise a pattern of stimulation signals, and the pattern of stimulation signals may be determined from muscle displacements sensed during the training set of motions. The muscle displacements may be sensed using one or more of an electromyogram sensor, a camera, an inertial motion unit, a bend/joint angle sensor, and a force sensor. The processor may perform the comparison using one or more of a support vector machine (SVM) algorithm, a hand-writing recognition algorithm, a dynamic time warping algorithm, a deep learning algorithm, a recursive neural network, a shallow neural network, convolutional neural network, a convergent neural network, or a deep neural network. The processor may perform the comparison using a Long Short-Term Memory type recursive neural network. The training set of motions may be performed by a second human. The training set of motions may be performed by the human using a laterally opposite body part of the first body part. The motion sensor may be located on an arm of the human and the muscle stimulator may be adapted to stimulate muscles to move one or more fingers of a hand of the human to perform a grasping motion. The expected trajectory may be in the shape of an alphanumeric character.

According to one embodiment the device comprises an orientation sensor connected with the processor and adapted to monitor an orientation of the first body part. A force applied by the grasping motion may depend on an amplitude of the stimulation signal and the processor may adjust an amplitude of the stimulation signal based, at least in part, on an output of the orientation sensor. The processor may adjust the grasping motion to be a key grip, a cylindrical grasp, or a vertical pinch in response to the output of the orientation sensor. The device may comprise a camera connected with the processor and positioned proximate to the hand to capture an image of an object to be grasped. The processor may adjust the grasping motion based in part on the image. The processor may comprise a close delay timer and the processor may delay stimulating the grasping motion for a predetermined period at the end of the actual trajectory determined by the close delay timer. The processor may cause stimulation of the hand to perform a post-grasp activity in response to a post-grasp signal from the motion sensor. The post-grasp activity may be opening the hand to release the grasp. The post-grasp signal may be one or more taps of a grasped object against a surface.

According to another embodiment a device is disclosed comprising one or more motion sensors, the sensors generating one or more respective motion signals indicative of motion of a first body part of a human, a muscle stimulator, the stimulator generating a stimulation signal adapted to cause or to increase a contraction of a first muscle, wherein the first muscle is a neurologically injured muscle, a paralyzed muscle, a partially paralyzed muscle, or a healthy muscle, and a processor connected with the sensor and the muscle stimulator. The processor includes data storage, the data storage including at least one expected trajectory associated with an intention of the human contract the muscle. The processor receives the one or more motion signals from the one or more sensors, calculates an actual trajectory of the first body part, compares the actual trajectory with the expected trajectory, determines the intention to contract the muscle based on the comparison, and causes the stimulator to do one or more of cause the contraction of the first muscle, assist the contraction of the first muscle, and cause an antagonist contraction of a second muscle, where contraction of the second muscle opposes a movement caused by the contraction of the first muscle. The device may comprise a nerve stimulator connected with, and operable by the processor and in response the processor determining the intention to contract the first muscle, the nerve stimulator may apply a nerve stimulation signal to a nerve of the human. The nerve of the human may be selected from one or more of a vagus nerve, a trigeminal nerve, a cranial nerve, a peripheral nerve feeding the first muscle, and a spinal cord of the human. The nerve may be the spinal cord and the nerve stimulator may comprise a transcutaneous electrode positioned above, over, or below a spinal cord injury of the human.

According to one embodiment a device is disclosed comprising one or more motion sensors, the motion sensors generating one or more respective motion signals indicative of motion of a first body part of a human, a prosthetic appendage comprising an actuator adapted to change a configuration of the prosthetic appendage to perform an action, and a processor connected with the one or more motion sensors and the actuator. The processor includes data storage, the data storage including at least one expected trajectory associated with an intention of the human to perform the action. The processor receives the one or more motion signals from the one or more motion sensors, calculates an actual trajectory of the first body part, compares the actual trajectory with the expected trajectory and, based on the comparison, actuates the actuator to change the configuration of the prosthetic appendage to perform the action. The prosthetic appendage may comprise a prosthetic hand and the actuator may comprise one or more of a wrist actuator and a finger actuator.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows a person's arm and hand equipped with a device according to an embodiment of the disclosure performing a test to measure finger dexterity;

FIG. 2 is a block diagram of a system according to one embodiment of the disclosure;

FIG. 3 shows the position, velocity, and acceleration of the person's arm equipped with the device as shown in FIG. 1 when the person moves his arm along a “C”-shaped path of motion;

FIG. 4 shows the position, velocity, and acceleration of the person's wrist equipped with the device as shown in FIG. 1 when the person moves his arm along a “number 3”-shaped path of motion;

FIG. 5, shows a system according to embodiments of the disclosure integrated into a wearable patch;

FIG. 6 shows a person's arm and hand equipped with a device according to an embodiment of the disclosure transferring a pen from one location to another;

FIG. 7 is a graph showing the performance of apparatus according to embodiments of the disclosure in identifying a patient's limb motion with a predefined trajectory;

FIG. 8 shows a comparison of confusion matrices for embodiments of the present disclosure using different machine learning algorithms to identify predefined trajectories; and

FIG. 9 shows a prosthetic limb including a device according to an embodiment of the disclosure.

### DETAILED DESCRIPTION

Some patients who have suffered neurological injury, such as a stroke or spinal cord injury have lost the ability to control motion in one part of their body but retain the ability to move other body parts. In some cases, the residual limb motion may allow the patient to move their shoulder and upper arm and to flex their elbow while the ability to control the motion of the hand, for example, to grasp an object, is lost. In other cases, a patient may have lost the ability to articulate their knee and ankle, while they retain residual motion of their hip. In the case of amputees, a patient may retain complete function of the residual portion of the amputated limb.

A system according to embodiments of the present disclosure senses and recognizes—through machine learning methods—residual limb trajectories and body motions in space and discerns the intention of the user to perform a specific action. Using sensors on the arms, legs, and/or body, a wide variety of two- and three-dimensional (2D/3D) motions, including translational, rotational or combinations thereof, can be recognized. The system includes circuitry that delivers LAMES signals to muscles controlling motion of the disabled body part or operates a robotic/prosthetic limb to restore hand/arm or foot/leg control.

According to a further embodiment, the system detects the fluid, natural, curvilinear path of motion of the functional body part normally associated with a desired action and causes the disabled body part to execute the action. For example, in a patient that has residual motion in his or her shoulder and upper arm, the device recognizes reaching trajectories and causes the patient's disabled hand to open and close to grasp an object. As used herein, the term “trajectory” means general motion of a body part including translational and/or rotational motion of the body part in space, as well as angular displacement of the body part about a joint (e.g. deflection of the elbow, shoulder, hip or knee).

Different reaching trajectories can be detected and, in response the system positions the patient's hand appropriately for that type of reach. For example, where the patient moves their arm and shoulder forward, or in a curvilinear pathway, with the wrist in the neutral, “handshake” position, the system discerns that they intend to grasp a vertically oriented object like a glass or water bottle resting on a tabletop (a “cylindrical grip”) by comparing the actual trajectory of the arm or shoulder with an expected trajectory associated with patients intent. In response to the discerned intention, the system energizes LAMES electrodes on the patient's forearm to activate the appropriate muscles to cause the hand to open in preparation of grasping the object and then, after a delay, the system stimulates muscles causing the fingers to wrap around the object and hold it securely. Alternatively, where the patient uses the residual motion of their shoulder and arm to reach along a vertical “rainbow” arc, the system discerns that the user intends to pick up an object from above with a pinching hand motion (a “vertical pinch”). Also, a patient may reach for an object using a “corkscrew” motion to indicate their intention to perform a third type grasp, such as a “claw grasp” to pick up an object. The device actuates LAMES electrodes controlling the hand to cause the patients thumb and fingers to open and then come together around the top of the object. An advantage of using natural motions of the residual body part to control the disabled body part is that the patient's actions more closely match an able-bodied person. This can draw less attention to the user and may promote neuroplasticity and rehabilitation in a stroke patient or recent spinal cord injury patient, for example.

The types of residual motion detected can also include predetermined trajectories that the patient executes, for example, movement of the arm along a “C”-shaped path. Just as a child traces letters, numbers, and patterns in the air with a sparkler, the device recognizes the pattern. The patient moves his able-bodied joint along the predetermined expected trajectory and the system discerns that a particular action is intended. In response, the system actuates LAMES electrodes that cause muscle contractions to execute the desired action. For example, a patient might execute a “C”-shaped motion with the shoulder and upper arm to cause the hand to open and close around a cylindrical object and an “S”-shaped motion to close the hand in a pinching motion. An advantage of using pre-programmed expected trajectories is that the number of specific motions that can be encoded is vast. The device can be programmed to recognize both pre-trained patterns and natural reaching trajectories. Moreover, new trajectories for new actions can be added to the patient's repertoire of actions.

According to one embodiment, the device energizes LAMES electrodes to stimulate the proper muscle contractions to execute the intended action. According to other embodiments the device recognizes motion paths of the patient's able body part to actuate prosthetic/robotic devices that facilitate movement in the paralyzed joints. In the arm, movement trajectories of the arm, driven by residual shoulder movements, can be used to drive stimulation or robotic control of a prosthetic hand adapted to perform multiple wrist, hand, and finger movements. Such a prosthetic hand includes a combination of wrist and finger actuators. In addition, certain motions can be detected to control external devices such as a computer, stereo, a motorized wheelchair, and the like. Because the number of distinct motion paths is quite large, the device can be used both to control a disabled body part, for example, using the natural trajectory of the shoulder in a reaching motion to control a disabled hand, and to control an external device like a computer using a pre-programmed motion path, (e.g., a “C”-shaped path).

Using devices according to embodiments of the disclosure to drive neuromuscular or robotic-driven movement in paralyzed joints may have additional assistive, rehabilitative, and therapeutic applications in stroke, spinal cord injury, and neurodegenerative conditions. Because the patient uses residual motion in the able-body joints, the patient strengthens the musculature and neural connections to perform that residual motion. In addition, as the device is used, brain plasticity associates the residual motion (both natural motions and pre-programmed motion paths) with the desired action, making the patient's motions appear more fluid like that of an able-bodied person. This approach also has application in general physical therapy after injury or surgery to the hand, foot, or other parts of the body. Furthermore, the disclosure herein can be used to measure and track the quality of limb/body movement trajectories over time in rehabilitative applications.

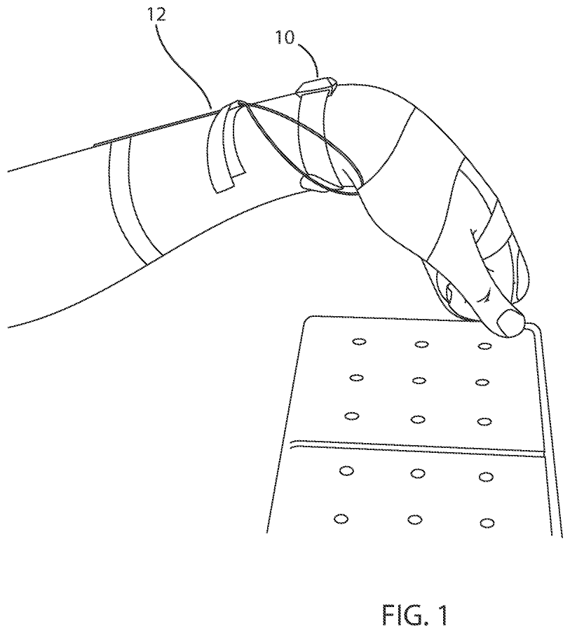

FIG. 1 shows the hand and forearm of a patient equipped with a device according to an embodiment of the disclosure while preforming a “Nine-hole Peg Test,” a standard measure of hand dexterity known to those of skill in the art. At the top of the patient's wrist is a wearable sensor housing 10 that includes motion sensors to detect the path of motion of the patient's hand and orientation of the patient's limb. As will be explained more fully below, the sensors may include inertial motion units (IMUs) to detect three-axis acceleration, gyroscopic sensors to detect rotational velocity, and magnetic sensors to detect orientation in earth's magnetic field. According to other embodiments, sensors can also include joint angle/bend sensors to detect flexing of a joint such as the elbow, knee, or hip. A computer (or microprocessor embedded in the device), not visible in FIG. 1, is in communication with the IMU. The computer includes a processor, memory, and/output devices. According to the embodiment shown in FIG. 1, the IMU communicates with the computer via a radio frequency Bluetooth link. LAMES electrodes 12 are in contact with the patient's abductor pollicis brevis and flexor pollicis brevis in this test to govern basic movement of the thumb.

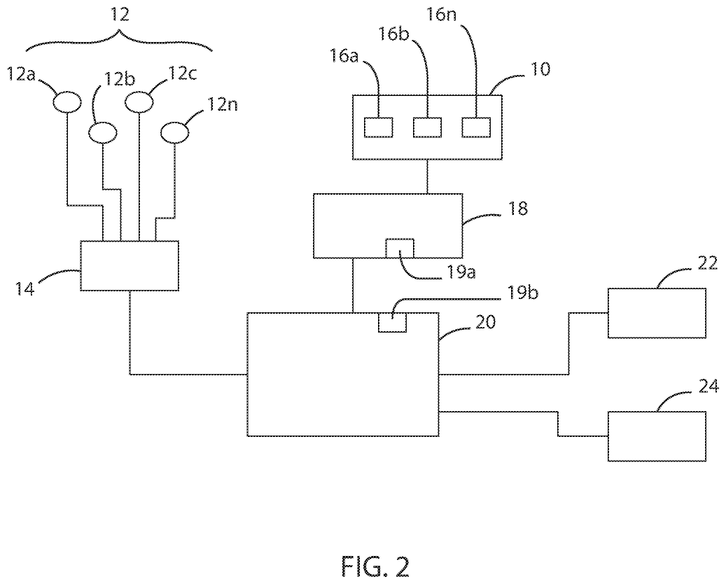

FIG. 2 is a block diagram illustrating an embodiment of the system in FIG. 1. Sensor housing 10 includes sensors 16a, 16b, . . . 16n. These may include IMUs, joint bend/angle sensors, cameras, gyroscopic sensors, force sensors, as well as other sensors for monitoring motion and orientation. A microcontroller 18 is connected with the sensors to preprocess signals from the sensors to integrate outputs from various sensors to provide trajectory data such as body part orientation, 3-axis linear acceleration corrected for gravity, or general motion (translational and/or rotational) information. Output from microcontroller 18 is provided to computer system 20 to provide signals indicating the path of motion of the patient's hand and analyze that motion, as will be described below. According to one embodiment, microcontroller 18 and computer 20 include radio frequency transceivers 19a and 19b, such as a Bluetooth or ZigBee protocol devices to communicate motion data wirelessly. According to other embodiments, the functions of computer 20 may be integrated into the microcontroller 18. This microprocessor can also be a neural processor or neural processing unit or tensor processor optimized for machine learning or deep learning consuming low levels of power, making it ideal for wearable devices (examples include the M1 processor by Apple (Cupertino, Calif.) or Cortex-M55 by Arm (Cambridge, England). Computer 20 may also include a network of computers connected locally and/or computer systems remote from the wearer, such as cloud computing systems.

According to the embodiment shown in FIG. 1, sensor housing 10 is worn like a wristwatch. Other types of housing could also be used. For example, the sensor housing 10 could be built into a cuff, sleeve, or wearable adhesive patch (with electrodes, microprocessor or artificial neural network or AI processor, visual indicators such as LEDs, wireless communication, and disposable conductive adhesive material) on the patient's forearm, or a glove worn over the patient's hand. Such a sleeve or wearable adhesive patch may incorporate a joint bend/angle sensor to detect flexing of the patient's elbow. For applications where residual motion of other body parts controls actuation of a disabled limb or external device, the device could be worn as a belt (to detect hip motion), as part of a hat or headband (to detect motion and orientation of the patient's head), or built into an article of clothing worn elsewhere on the patient's body.

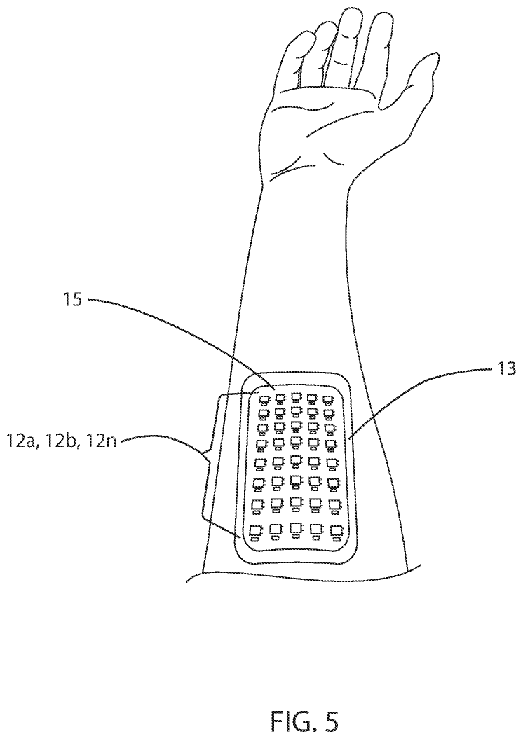

Computer 20 is connected with an NMES driver 14 that generates currents to apply to a plurality of NMES electrodes 12a, 12b, 12c, . . . 12n. The NMES electrodes are placed on the patient's forearm or are incorporated into a cuff, sleeve, or adhesive patch. According to one embodiment, NMES electrodes 12a, 12b, 12c, . . . 12n are arranged in a sleeve that fits securely onto the patient's forearm as shown in FIG. 5 and discussed in detail below. The arrangement of electrodes is selected to correspond with the muscular anatomy of the forearm. Once in place, the NMES electrodes may be mapped to the patient's musculature.

NMES driver 14 generates stimulation waveforms that are applied to selected sets of electrodes. Parameters for the waveform, including waveform shape (square, sinusoidal, triangular, or other), pulse-width, pulse frequency, voltage, and duty cycle, are selected and the LAMES driver is set to apply these signals in response to control signals from computer 20. According to one embodiment, stimulation is applied as a series of brief bursts separated by an inter-burst period. LAMES parameters may be selected to improve penetration through the skin, to more precisely isolate finger and thumb movements, and to reduce fatigue. The electrodes are mapped to specific muscles in the patient's forearm so that the stimulation signals from the LAMES driver activate selected muscles to activate fingers and thumb flexion and extension.

In the example shown in FIG. 1, LAMES electrodes are applied to the patient's forearm using adhesive tape or an adhesive conductive material or hydrogel. Alternatively, electrodes could be built into a patch (with disposable adhesive hydrogel) or cuff with integrated sensors and microprocessor or AI processing unit worn over the patient's forearm as shown in FIG. 5 and discussed below. Other methods of connecting and orienting electrodes relative to the patient's musculature know to those of skill in the art may be used. In the embodiment shown in FIG. 1, electrodes 12a, 12b, 12c, . . . 12n are arranged to apply a stimulation current to one or more of the thumb muscles controlling the thumb (the abductor pollicis brevis, flexor pollicis brevis, and opponens pollicis), which evoke various useful thumb movements including “pinching” (with tip of index) and “key” style grasping.

Computer 20 includes hardware and software components for receiving signals from sensors 16a, 16b, . . . 16n to determine the trajectory and orientation of housing 10, and hence, the path of motion and orientation of the patient's limb. Based on this, computer 20 sends signals to the LAMES driver 14 to energize electrodes 12a, 12b, 12c, . . . 12n according to a sequence that causes the patient's hand to assume the intended configuration. According to one embodiment, computer 20 also provides output to an output device 22 such as a display monitor or screen and receives input from one or more input devices 24, such as a keyboard, a computer mouse or other pointing device, and/or a microphone. Output from the computer may also be recorded and used by medical professionals to assess the patient's progress during physical therapy. In addition, as will be discussed more fully below, the output may be anonymized and collected, along with similar data from a population of patients and used to train machine learning systems to better recognize body motions and trajectories that indicate the intention of a user to perform the intended action.

According to another embodiment, computer 20, NMES driver 14, microcontroller 18, and the array of NMES electrodes 12a, 12b, 12c, . . . 12n are integrated with the sensor housing 10 to form a portable, wearable system. Such a wearable system might include a touchscreen or other input/output device similar to a “smart watch” to allow the patient to interact with the system, for example, to train the system to better discern the patient's intentions. Connections between computer 20 and other components of the system may be a physical connection, e.g., cables. Alternatively, computer 20 may communicate signals wirelessly by a radio frequency link (e.g., Bluetooth, ZigBee) or via infrared. The computer 20 includes memory storage and is programmed to perform various algorithms, as will be described more fully below. According to other embodiments, computer 20 is also integrated into sensor housing 10. Such an embodiment provides a self-contained system allowing the wearable system to be used independently from any wired or wireless interface.

FIG. 5 shows an embodiment of the disclosure with an array of NMES electrodes 12a, 12b, . . . 12n integrated on a wearable patch 15. An electrical coupling layer 13, such as a hydrogel layer is provided between the electrode array and the wearer's skin. In the embodiment shown in FIG. 5, electrodes 12a, 12b, . . . 12n are arranged in a pattern adjacent to the musculature controlling the wearer's hand. According to one embodiment, other components, such as sensor housing 10 including IMU sensors 16a, 16b, . . . 16n, microcontroller 18, NMES driver 14, computer 20, and a power source are also disposed on wearable patch 15. This embodiment eliminates cabling, allowing the user to freely move the able-bodied joint, in this case the shoulder, torso, and upper arm, or hip, to actuate the system to stimulate intended actions in the disabled joints of the hand, lower leg, or foot. Eliminating cabling enables the device to be worn continuously to assist the user with daily activities. Electrode array 12 may be programmed to map particular NMES drivers 12a, 12b, . . . 12n to the wearer's musculature so that energizing specific electrodes or sets of electrodes results in particular motions, for example, grasping motions, as described above, or lower leg, or foot. Such mapping may use machine learning techniques to fine tune the activation of muscles to the intentions of the wearer.

In the example shown in FIG. 1, inertial sensors (IMUs) 16a, 16b, . . . 16n in housing 10 on the patient's wrist sense 2D and 3D arm trajectories and send signals to computer 20. These signals are analyzed and compared with one or more expected trajectories associated with a desired action using data fitting and/or machine learning algorithms running on computer 20. When a trajectory or motion indicating that the patient intends to perform a particular action is recognized, computer 20 sends signals to the LAMES driver 14 to activate selected electrodes 12a, 12b, 12c, . . . 12n to control neuromuscular stimulation patterns in the forearm to control the hand to “open” and “close.”

The IMUs monitor the actual trajectories of the patient's limbs and provide signals that are analyzed to indicate desire movements or device actions. The IMUs may detect 6-axis (acceleration and rotational velocity) or 9-axis (adding magnetic field information) motion. One or more housings with IMUs can be placed on various limb, body, or head locations and used to provide orientation and translation information for the patient's limb segments in the leg, hand, foot, hip, neck, head, or any other body part.

When the patient shown in FIG. 1 moves his hand in a vertical “rainbow” arc, output of the IMU attached to his wrist (or forearm) is analyzed by computer 20 to detect this as an expected trajectory and discern that he intends to grasp a peg from the pegboard using a “key grip” type motion. As the patient's hand nears the end of the vertical arc trajectory, computer 20 causes LAMES driver 14 to stimulate the patient's muscles to curl the fingers of the hand and to move the thumb away from the index finger so that the thumb is extended and prepared to assume a “key grip” on the peg. When the hand reaches the end of the vertical arc trajectory, computer 20 causes the thumb to remain spaced away from the side of the index finger for a time delay to allow the patient to position the hand with respect to the peg using his residual shoulder and arm function. At the end of the delay, computer 20 actuates the NEMS electrodes over the extensor pollicis brevis muscle thus closing the grip on the peg. NEMS signals remain active so that the peg remains securely gripped. Other general motions (i.e., translational and/or rotational motions) of the patient's wrist or forearm could be sensed to determine the patient's intention to perform other types of grasping motions. For example, the patient may reach for an object using a “corkscrew” motion to indicate their intention to perform another type of grasp, such as a “claw grasp” to pick up an object.

Computer 20 keeps the muscles activated until the patient performs another motion or trajectory indicating that the patient wishes to release his grip. According to one embodiment, when the patient moves their hand (using residual shoulder/elbow movement) in a small clockwise or counter-clockwise motion in the horizontal plane parallel to the table's surface the motion is detected by an accelerometer, for example, one or more of the IMU sensors 16a, 16b, . . . 16n. This motion is interpreted by computer 20 as indicating the patient's intent to release the peg. The computer 20 causes NMES currents to be applied to move the thumb away from the forefinger, opening the grip and releasing the peg. Other motions could be used to indicate that the object should be released, such as a pronation or supination (rotation) type motion of the forearm. The user may select any pattern of motion or body movement to indicate the intent to release the grip, which can be identified to the pattern recognition and/or machine learning algorithms to evoke a “hand open” neuromuscular stimulation pattern. According to another embodiment, instead of, or in addition to, a body motion or trajectory, the signal that the patient intends to release the object is an abrupt signal, such as tapping the object on a surface one or more times, thereby generating an accelerometer signature signal. A tapping signal may be particularly advantageous when a cylindrical object such as a water glass is grasped because tapping can be done subtly, so as not to draw attention to the person's disability. A simple clockwise or counterclockwise circular motion in the horizontal plane can also be used to indicate the user desires to open their hand and release the object.

According to another embodiment, instead of sending signals to an NMES driver, computer 20 is connected with motorized actuators of a robotic/prosthetic hand that replaces a patient's amputated hand. In this embodiment, the robotic hand is controlled to perform grasping actions in response to the detected arm trajectory.

According to one embodiment, the interpretation of a trajectory depends on the state of the system prior to detecting the trajectory. In the example just given, in the state where an object has been grasped, the clockwise circular motion/trajectory in the horizontal plane is interpreted as a command to release the object. When the system is in a different initial state, for example, when the hand is in an “open” position, a clockwise circular motion might cause a different action, for example, to perform a claw grasp.

Embodiments of the disclosure are not limited to detecting motion of the hand or arm. The human body can achieve an infinite number of motions in space as we move our limbs and trunks in various patterns. Specifically, the rotation and trajectory in space of our arms and legs, and even hips and trunk, contain a vast amount of information. Disclosed here are methods and devices to sense and recognize a variety of movements to achieve various desired outcomes in a robust accurate way. Natural reaching movements (using residual shoulder movement) can be described by specific straight or curved motions in space, sometimes accompanied by limb (or body) rotation as well. For example, with this approach a quadriplegic user can move their arm along a curved path towards an object and this trajectory will be automatically recognized and subsequently trigger neuromuscular stimulation causing their hand to open and then close (after a short delay) around an object.

An IMU can also provide orientation information which can be very useful. If, for example, the IMU is located on the back of the wrist (forearm side of the wrist where a watch face would be located), and the hand is in a neutral (handshake) position, this information, combined with a specific reaching trajectory can indicate the user desires to grasp a cylindrical object such as water bottle or glass. 2D arm trajectory and/or orientation patterns can be used to drive a large number of actions including device control and muscle stimulation patterns for various hand/leg movements. Furthermore, various trajectories can be used to control different types of grasping. As discussed above, a rainbow-like arc trajectory, as a user reaches out and over the top of an object lying on a table, could trigger a claw type open and close grasp for picking up that object from above. A clockwise-corkscrew type reaching trajectory could be used to control a cylindrical grasp, while a counter-clockwise corkscrew reaching pattern could be used for a pinch-type grasp.

In addition to IMUs, other sensors can be used to detect motion of able-bodied joints. According to one embodiment, a bend sensor is provided at the elbow to provide additional input. This input can be used to further identify a particular trajectory. Elbow bending may also be used to modulate the neuromuscular stimulation current amplitude for driving grasp strength during gripping actions (or the closing force of a robotic end effector).

According to another embodiment of the disclosure, instead of, or in addition to detecting natural body motions, the device detects one or more predefined trajectories. Just as one moves a sparkler in the air, recognizable patterns and shapes can be generated (e.g., letters, numbers, corkscrew/spiral, etc.). Sensors 16a, 16b, . . . 16n detect motions associated with such patterns and computer 20 analyses the signals form the sensors to determine if the patient has executed a pattern that corresponds to a particular action. The user can select any patterns they prefer and link it to various movements or device actions (home electronics, computer, mobile device, robotic arm, wheelchair, etc.). These trajectories can be used to interact with, control, or drive these devices under direct user control.

A device was constructed according to embodiments of the disclosure. Sensors 16a, 16b, . . . 16n consisted of a Bosch SensorTec BNO055 9-axis IMU. The sensor was connected with a microcontroller 18, here a 32-bit ARM microcontroller unit (MCU) from Adafruit (Feather Huzzah32). The IMU has a built-in processor and algorithms to estimate its orientation and perform gravity compensation in real-time to produce linear acceleration in three orthogonal directions. Linear acceleration along the X, Y, and Z axes was available externally via an I2C interface. A flexible printed circuit board was designed to interconnect the IMU with the MCU 18. Data was continuously streamed from the MCU at 50 Hz via Bluetooth to a computer 20. Computer 20 used MATLAB 2019a to store and process motion data for embodiments where processing was performed offline.

In other embodiments, MCU 18 performed data processing in real-time to actuate muscle stimulators positioned on a test subject's forearm. Neuromuscular stimulation was provided by a battery-operated, 8-channel, voltage-controlled stimulator, with a stimulation pulse frequency of 20 Hz. The stimulation channels were mapped to individual or multiple electrodes on a fabric sleeve, in order to evoke various finger flexion and extension type movements. By grouping multiple stimulation channels and sequencing their activation profile, different grasp types such as cylindrical and pinch grasps were programmed.

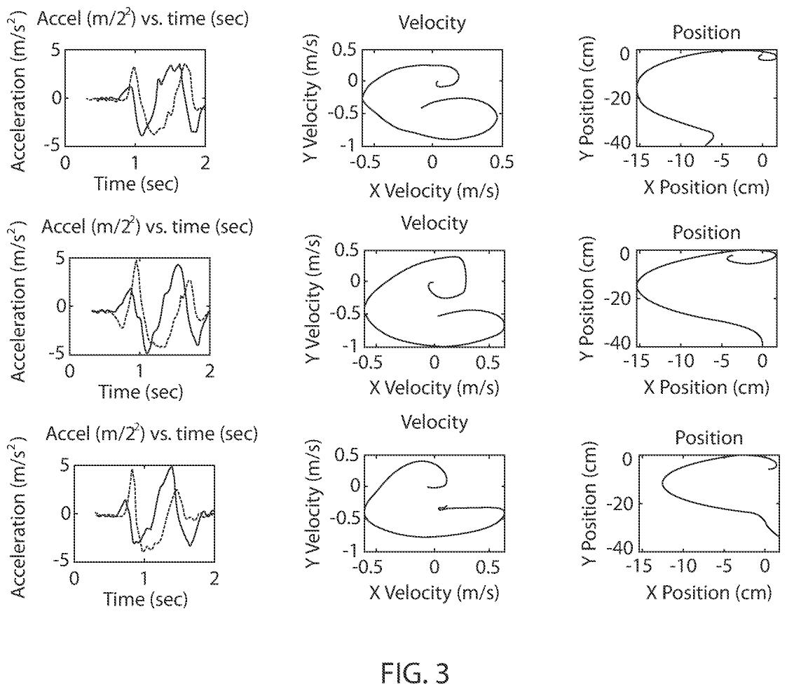

FIG. 3 shows motions recorded by a device according to a further embodiment of the disclosure. In this example, an able-bodied person wearing a device according to an embodiment of the disclosure moved his arm along a “C”-shaped trajectory. In this example, the person repeated the motion three times. Signals from IMU provided 6-axis data (acceleration and rotational velocity) of the persons wrist. The output of the IMU is corrected for gravity to provide repeatable acceleration data that is integrated to determine the time-dependent position (i.e., the trajectory) of the limb during the motion. Based on the trajectory, computer 20 determined that the “C”-shape trajectory was made. In each repetition, the “C”-shape is apparent in the X/Y position displayed in the right-most column of graphs.

Computer 20 may use pattern recognition algorithms to analyze and identify limb and body motions and trajectories to discern the patient's intention to perform an action. The analysis may include signal processing algorithms including Dynamic Time Warping (DTW) to compare the actual trajectory of a patient's limb motion with the trajectory expected to correspond to an intentional action. DTW has the advantage of being able to accommodate different motion/trajectory speeds or timing profiles that different users may have.

According to other embodiments machine learning techniques are applied to analyze the sensor output to discern the user's intention to perform a certain action and to distinguish other motions where the user does not intend an action to occur. According to one such embodiment, computer 20 includes a convolutional neural network (CNN) or recurrent neural network (RNN) to analyze data from IMUs and other sensors to identify body motions and trajectories that signal the patients intention to perform an action or camera data to provide additional contextual information to further discern the user's intentions or information about the object the hand is approaching (shape and size of the object the hand must accommodate and grasp). The RNN implements techniques such as Long Short-Term Memory (LSTM) to identify volition-signaling motions. Using such techniques, the system repeatedly and reliably identifies specific trajectories or body motions and actuates the patient's muscles or motorized prosthetic devices to perform the intended action. In addition, because sensor data is recorded, systems according to the disclosed embodiments can be continually trained to better identify the patient's intentions. Data from multiple patients, when properly anonymized, may be gathered and used to train the machine learning algorithm. Various other machine learning algorithms can be used to analyze and identify natural and pre-programmed trajectories. These include but are not limited to, support vector machine (SVM) algorithms, hand-writing recognitions algorithms, and deep learning algorithms. Such machine learning algorithms may be implemented locally on a computer 20 worn on the patient's person (e.g., built into to a prosthesis or connected with the sensor housing 10). Alternatively, or in addition to local processing, machine learning algorithms may be implemented on a computer system remote from the user, for example, on a cloud computing network. This allows systems and methods disclosed here to adapt as additional data is collected over time. Such algorithms may recognize a patient repeating a body motion to allow the algorithm to recognize a motion not accurately detected the first time.

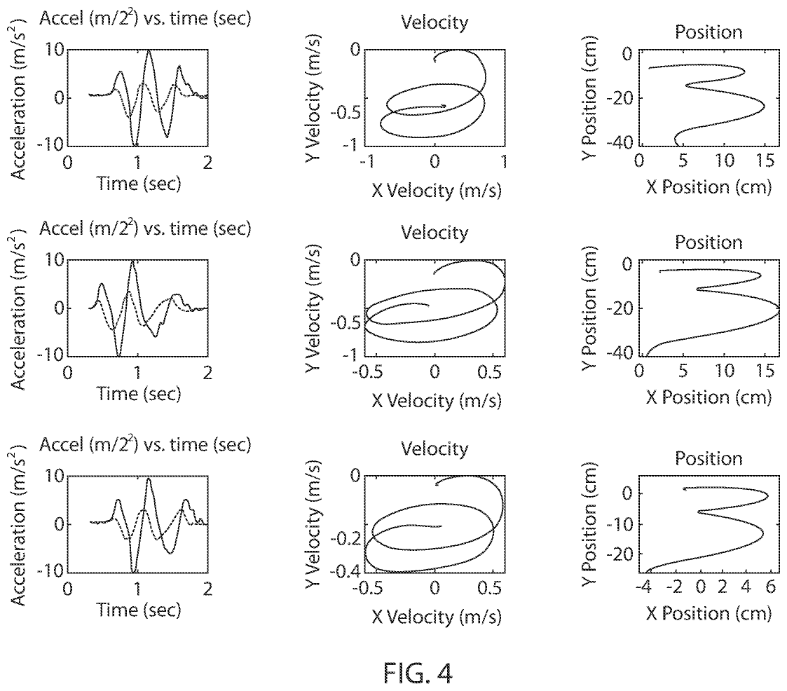

FIG. 4 shows another example of motion detection by a device according to an embodiment of the disclosure. Here an able-bodied person executed a “3”-shaped motion in three repetitions. Again, IMUs provided gravity-corrected acceleration data and the computer calculated the time dependent trajectory of the person's limb. Again, as shown by the position graphs in the right-most column, the “3”-shape was found in each repetition. Had a user associated the “3”-shape and the “C”-shape motion with a different actions, for example, a “key grip” and a “cylindrical grasp,” computer 20 could apply a different pattern of neuromuscular stimulation, resulting in hand motions to execute one or the other type of grasping.

According to another embodiment, training sets of motion data were prepared for various alphanumeric-shaped trajectories. First, the raw 3-axis gravity compensated acceleration obtained from the IMU was band-pass filtered (Butterworth, 8th order, 0.2-6 Hz) and processed offline for identifying training samples. The absolute value for the acceleration along the 3-axis was used to identify onset of movement by setting a threshold of 0.95 g. The movement onsets were then used to segment the acceleration data over time along the X, Y, and Z axis into windows ranging −0.1 s to 0.9 s with respect to onset. Each trial was visually confirmed to be free from any noisy artefacts or excessive jerk (derivative of acceleration) or if it exceeded the is window and such trials were excluded from further analysis. These training sets were used to train Dynamic Time Warping (DTW) and Long Short-Term Memory (LSTM) network algorithms.

The DTW algorithm optimally aligns a sample trajectory with respect to a previously determined template trajectory such that the Euclidean distance between the two samples is minimized. This is achieved by iteratively expanding or shrinking the time axis until an optimal match is obtained. For multivariate data such as acceleration, the algorithm simultaneously minimizes the distance along the different dimensions using dependent time warping. According to this embodiment, the algorithm was used to compute the optimal distance between a test sample and all the templates associated with the 2D and 3D trajectories. The template with the least optimal distance to the test sample, was selected as the classifier's output. Since the classifier's output is dependent on the quality of its templates, an internal optimization loop was used to select the best template trajectory from a set of training trajectories. Within this loop, the DTW scores of each training sample with every other training sample was computed. Then the training sample with the least aggregate DTW score, was chosen as the template for that trajectory, that is, the expected trajectory.

In some embodiments, an LSTM network is used to analyze motion data. According to one such embodiment the LSTM network comprised of a single bidirectional layer with 100 or more hidden units provided with the MATLAB R2019b Deep Learning Toolbox. Default values were selected for most parameters. The LSTM network transformed the 2D or 3D acceleration data into inputs for a fully connected layer whose outcome was binary, i.e. 0 or 1. Next, a softmax layer was used to determine the probability of multiple output classes. Finally, the network output mode was set at ‘last’, so as to generate a decision only after the final time step has passed. This allowed the LSTM classifier to behave similar to DTW and classify trajectory windows. During training of the LSTM network weights, adaptive moment estimation (ADAM) solver was used with a gradient threshold of 1 and maximum number of epochs of 200. Since all the training and validation data were 1 second long, zero padding was not used. To implement the LSTM network, a MATLAB R2019b Deep Learning Toolbox was used with default values for parameters other than the ones mentioned above.

According to one embodiment, online classification of arm trajectories was performed by filtering and processing the raw acceleration signals in real-time using a MATLAB script that looped at 50 Hz. Within the loop, the acceleration data was divided into 1-second long segments with 98% overlap. The DTW-based classifier was implemented and was designed to compare the incoming acceleration windows with 2D trajectories. If the optimal distance between trajectories were below 10 units (empirically determined), then positive classification was issued, which then triggered the LAMES driver 14 to stimulate muscles to perform a complete movement sequence of opening and closing of the hand.

According to one embodiment sensor data is input into a machine learning algorithm that is trained to identify particular motions as expected trajectories to associate with actions. Such training may be accomplished by using able-bodied persons or the unaffected side (mirror image of the movement) in a stroke patient. In a stroke patient, hemiplegia (paralysis on one side of the body) is very common. According to one embodiment, the stroke user uses their unaffected side to train the device's algorithms, or further tailor to, their movements. In either case, the user wears the device while performing natural reaching and various trajectories under real-world conditions with an additional sensor detecting hand opening and differing grasping actions. Such additional sensors include EMG (electromyogram) sensors placed over the related muscles to determine the hand grasping actions (open, close, key grip, cylindrical grip, etc.). The amplitudes of this EMG signal represent the muscle contraction strength, including its duration and change over time, and this data can be used directly to inform the electrical stimulation amplitudes, and their timing, applied by the NMES driver 14 to deliver a pattern of stimulation signals to perform the grasping action when a desired movement is recognized through motion/trajectory recognition algorithms. The device for detecting hand opening and differing grasping actions may also include a camera for recording images of such actions, an IMU, or joint angle/bend or force sensor attached to the able-bodied hand used train the system to determine the pattern of stimulation signals.

Additional sensors may also include a camera coupled with image analysis and positioned to capture the reaching trajectory and/or grasping motion as well as bend/joint angle and force sensors. Captured trajectory and grasping data is used to build a database of pre-trained trajectory or motion patterns to be associated with certain hand actions. This data is used to train a machine learning algorithm such as a deep learning neural network. The device may be trained (or partially trained) before the device is fitted to a disabled person. Such training may include the use of inputs to computer via input devices 24. For example, a person training the system to recognize a particular trajectory as an “S”-shaped path that indicates a cylindrical-type grasp may audibly say words such as “cylindrical grasp,” “open,” and “closed” in synchrony with the motion. The computer 20, using a microphone as input and known voice recognition techniques, reads the audio input and tags the sensor data. This tagged data becomes part of the training set of data for the machine learning algorithm. Alternatively, motions used to train the algorithm may be tagged using keystrokes on a keyboard, or computer 20 may be equipped with a camera that captures visual images of the user performing various tasks (e.g., grasping objects on a table, inserting pegs into a board) while recording motion data from IMUs to associate “natural” grasping motions with the associated hand motion.

In another embodiment, a camera is located at the wrist (as part of a band, sleeve/patch, or clothing) to recognize objects as they are approached, thereby affecting the stimulation patterns to change the type of hand opening style (e.g. all fingers or just thumb-index pinch extensors activated) and when relative position of object to the hand slows down/stops, then flexors are automatically activated to initiate the grasp. Techniques for real-time object recognition using small portable devices using battery-powered microprocessors (e.g., cell phone technology) are well-known in the art. These techniques, combined with AI and machine learning methods such as support vector machines, convolutional neural networks, and long short-term memory (LSTM) recurrent neural networks for static and dynamic image classification allow visual cues, such as the type of object being grasped, to inform the system how to position the patient's hand to correctly and reliably grasp an object.

When considering 2D and 3D motions (e.g. corkscrew movements in the air), a large variety of trajectories may be identified by the computer and associated with various actions. These trajectories can not only be used to drive neuromuscular stimulation to restore movement, but also can be used to drive prosthetic/robotic devices or mobility devices like wheelchairs.

A study was performed using a system according to the disclosure to detect motion trajectories corresponding to selected predefined trajectories. Two participants with quadriplegia were recruited for the study. Participant 1 was a 32 year-old male, injured 6 years prior, with a C4/C5 ASIA (American Spinal Injury Association) B injury. He participated in 10 sessions, out of which 7 sessions were used to record 2D and 3D arm movement trajectories. During the remaining 3 sessions, grasping intentions were decoded online (in real-time) and used to drive a custom neuromuscular stimulator with textile-based electrodes 12a, 12b, . . . 12n housed in a sleeve. This in turn allowed the participant to perform functional movements (e.g. eat a granola bar). Participant 2 was a 28 year-old male, injured 10 years prior, with a C4/C5 ASIA A injury. He participated in 3 sessions, which involved 2 training and 1 online testing session.

Participants were seated with their hands initially resting on a table. A wireless sensor module was attached to the wrist of their arm using a Velcro strap. The sensor module included a motion sensor 16a, 16b, . . . 16n and an MCU 18, as disclosed in previous embodiments. While both participants were bilaterally impaired, each still possessed residual movement that allowed reaching with at least one of their arms and was eventually used for the study.

During the study, verbal cues associated with different 2D and 3D movement trajectories were randomly called out to the participant. The participants were instructed to perform the reaching trajectories starting from the edge or corner of the table and move towards the center, using smooth movements that were up to a second long. Three different 3D reaching trajectories: a sideways arc, a vertical arc (e.g. reaching for a pen or marker lying on a table), and a corkscrew motion were trained. Additionally, four 2D trajectories (performed in the horizontal plane) corresponding to well-known English and Greek letters: [symbol] , ε (epsilon or E), γ (gamma), and [symbol] were trained. Experiments were conducted in blocks of 18-20 trials and sufficient breaks were given between blocks to minimize participant fatigue. Initially, the participants were asked to perform only [symbol] and ε trajectories because these were simple to learn and didn't cause fatigue. Later, once the participants became comfortable with moving their arm, additional 2D and 3D trajectories were added to the study. Thus, in the final datasets there was a higher percentage of 2D trajectories (especially, [symbol] and ε) than the remaining trajectories.

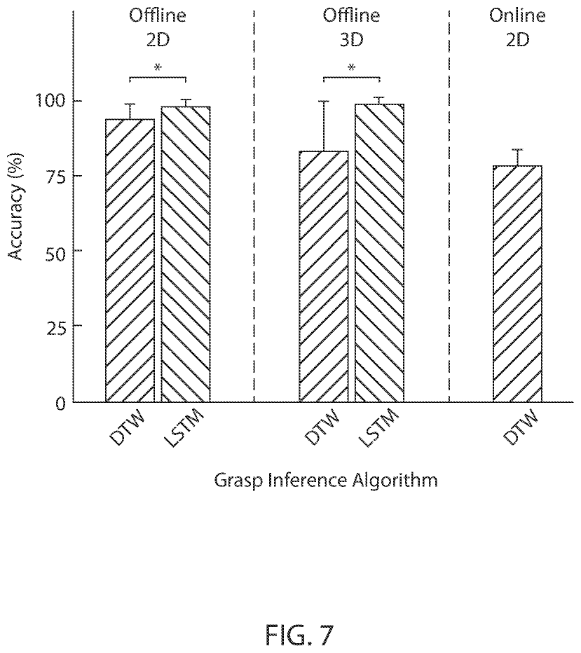

Over 250 training samples across 7 movement trajectories were recorded for participant 1 and 96 samples from 5 movement trajectories were recorded for participant 2. Trials with noisy sensor data or incorrect labels were visually identified and removed from the training set. A 5-fold stratified cross-validation scheme was selected for evaluating DTW and LSTM based classifiers. FIG. 7 shows the mean±standard deviation (SD) classification accuracy for the 2 participants. Bar graphs compare classification accuracies (Mean±SD) using two methods: DTW and LSTM. Performance was evaluated using both offline (2D & 3D) and online (2D only) arm trajectories. Statistical significance threshold was set at p<0.05.

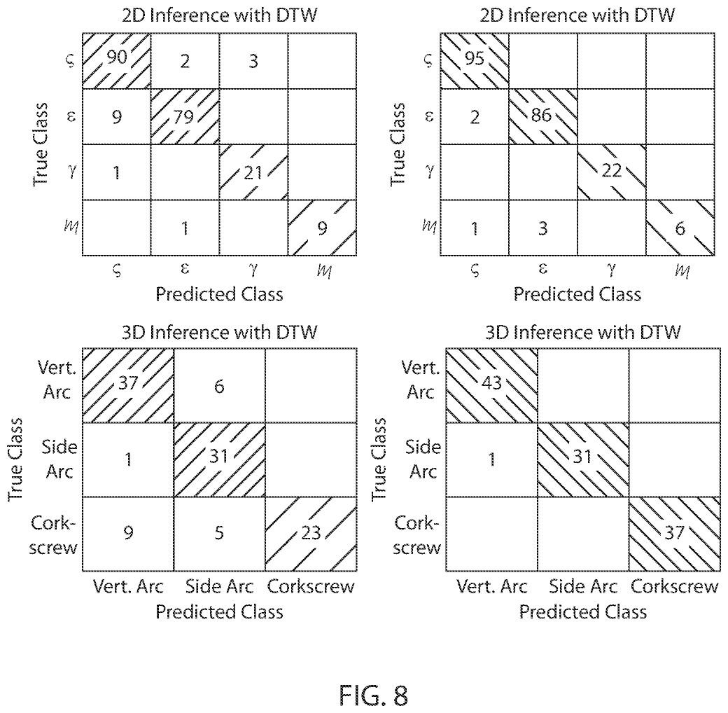

In the offline scenario both DTW and LSTM based classifiers performed well for 2D trajectories, achieving 94±5% and 98±3% accuracy, respectively. For offline 3D trajectories however, LSTM outperformed DTW and obtained 99±3% accuracy over 83±16%. Using two-sided Wilcoxon rank sum test, LSTM based classification accuracy was significantly better than DTW (p<0.05) in both cases. FIG. 7 also shows the online performance of DTW based classifier for 2D trajectories. During online classification, a comparison is made between 2 trajectories (e.g. [symbol] v/s ε) or between a single trajectory and rest (e.g. [symbol] v/s rest) and achieved 79±5% accuracy. To further evaluate each classifier's performance for type I and II errors, cumulative confusion matrices were calculated by adding the confusion matrices from each fold for each participant. The resulting confusion matrices for both classifiers and for both types of trajectories are shown in FIG. 8.

For DTW-based classifier, type I error occurred more frequently for 3D than 2D trajectories. The highest percentage of type I error occurred for the corkscrew trajectory (37.8%), followed by vertical arc (14%), (10.2%) and [symbol] (10%) trajectories. In terms of type II errors, DTW-based classifier misclassified vertical arc (14.5%), side arc (13.8%) and S (8.33%) trajectories as compared to rest of the classes. For LSTM-based classifier the type I and II errors were very low and ranged from 0-3% for almost all trajectories, with the exception [symbol] trajectory that had a type I error rate of 40%. It is surmised that because there were only 10 trials of [symbol] trajectory for training, this sample set was too small for the LSTM classifier to distinguish this trajectory from other classes that had larger number of samples.

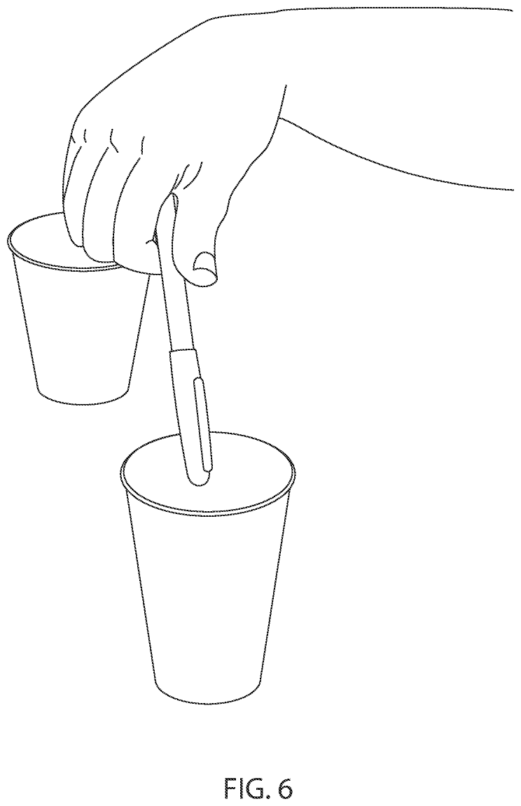

As a further example, a system according to an embodiment of the disclosure was tested by a paralyzed person with residual shoulder and arm motion, but without residual motion in his hand. As shown in FIG. 6, the device recognized the natural reaching motion of the person's arm and shoulder and stimulated the persons thumb adduction and abduction muscles to grasp a pen standing in one cup. The person was able to lift the pen using residual arm and shoulder motions and transfer it to a second cup while the device continued to activate the patient's muscles to keep a grip.

As another example, a system according to an embodiment of the disclosure was tested using an able-bodied person to predict muscle activation during a reaching and grasping motion based on training of an LSTM network using EMG signals. The subject was fitted with EMG sensors over the ring finger flexor and extensor muscles and an IMU 16a fitted to the wrist. Signals from the EMG and IMU were preprocessed with a microcontroller 18 implemented on a circuit board, an Arduino™ Nano 33 BLE. Data from the circuit board was wirelessly communicated to a computer 20 implementing an LSTM network, as described in previous embodiments. The subject performed repeated reaching and grasping motions while data from the IMU and EMG were provided to the LSTM network. After training the LSTM, the LSTM was able to predict the timing and amplitude of muscle activity of the flexor and extensor muscles based on the trajectory of the subject's wrist.

According to other embodiments, a device according to the disclosure can be used to enable movement of lower extremities. In one such embodiment, IMUs are affixed to a patient's hips. 2D and 3D hip movements are detected by analyzing data from the IMUs. Again, training the algorithms can be achieved by outfitting an able-bodied person with IMUs, cameras observing limb position and motion, bend/joint angle sensors in the leg joints and/or EMG sensors on the muscles to be stimulated in a paralyzed person. Hip movements can be used to actuate muscles using NMES, for example, to correct the person's gait or facilitate walking if they are weak, paralyzed, or have drop foot. During normal walking, the left hip/upper body traverses a 2D “C” shaped curved trajectory in space before the right leg is lifted. According to one embodiment, this tell-tale signature trajectory is recognized and used to trigger a muscle stimulation pattern in the right leg to assist with the stepping sequence. NMES electrodes may be placed over any muscle activating the joint of interest. For example, where a device according to the disclosure is used to facilitate rehabilitation following knee surgery, actuators may be placed over the quadricep, hamstring, calf, and foot extensor muscles to stimulate the muscles to encourage the wearer to perform an improved walking gait. Stimulation may be combined with the person using their arms to partially support their weight on a walker or parallel bars to assist their hip/upper body movement. The trajectory of the right hip is then detected and used to stimulate muscles of the left leg.

Systems according to embodiments of the disclosure may be integrated with gloves, shoes, and other garments that include force sensors. Such sensors detect contact and pressure applied between the wearer's hand and a grasped object or monitor the placement of the foot while stepping. Such garments may also include bend/angle sensors at the elbow, wrist, knee, ankle, or other joint to provide trajectory, orientation, and motion information to the system and/or data related to intention (during machine learning algorithm training in able-body users)

According to another embodiment of the disclosure, information about the trajectory, orientation, and position of the patient's limbs is collected by the system and recorded. Such information is used to track body part trajectories and/or joint movements (or ranges of motion) during physical therapy. Systems according to the disclosure provide a low-cost way for medical professionals to track progress and characterize motion (like gross arm movement in space) in rehabilitating a stroke or spinal cord injury patient. Such systems are less expensive and less cumbersome than current methods of monitoring limb position and motion that rely on expensive robotic systems or table sized devices. In addition, machine learning algorithms can compare a patient's movements with movements by able-bodied volunteers and other patients at various stages of recovery and grade or classify the patient's movements. This information may allow professionals to optimize therapies, provide patients with better feedback, and indicate progress of patient during their recovery.

During the training of user-specific (custom) trajectories, voice recognition, a brain-computer interface (BCI)—non-invasive or invasive (EEG), a touch pad, and/or able-bodied hand/leg motion can be used to initiate the training or select the desired action or hand or foot movement to be associated with the trained trajectory. Furthermore, pre-trained trajectory profiles can be stored in the device/system so that no training will be required. For example, letters, numbers, and patterns that are already known by the user can be available and automatically recognized without user-specific training.

According to another embodiment, instead of, or in addition to stimulating muscles to perform an action in response to a recognized trajectory, the system can also apply therapeutic stimulation elsewhere in the patient's neurological system. According to a still further embodiment, one or more of electrodes 12a, 12b, 12c . . . 12n are adapted to apply a stimulation current to the patient's peripheral nerves or to the patient's central nervous system (CNS) for a wide variety of applications including movement/sensory recovery and chronic pain. It is well known that neurostimulation can also be effective in treating pain through implanted and transcutaneous stimulation devices. It is also well known that certain types of movements (raising the arm or bending over at the waist) can cause pain. According to some embodiments of the disclosure, translational and/or rotational motion of a body part that might cause pain triggers stimulation to reduce pain caused by the detected motion.

Using motion/trajectory recognition to trigger various types of stimulators according to embodiments of the disclosure can have many benefits. For example, vagus nerve stimulation has been shown to improve the efficacy of upper limb rehabilitation. According to one embodiment, the system triggers vagus nerve stimulation cervically (neck) or auricularly (ear) during movement rehabilitation for stroke, SCI, traumatic brain injury, MS, etc. Such therapeutic stimulation can be applied to other nerves, such as the trigeminal nerve and other cranial nerves or peripheral nerves feeding muscles of interest. Systems according to the disclosure can also be used to trigger, control, and and/or modulate various forms of brain stimulation including TMS (transmagnetic stimulation) and tDCS (transcutaneous direct-current stimulation), tACS (transcutaneous alternating current stimulation), TENS (transcutaneous electrical nerve stimulation), or spinal cord stimulation (which sends signs down the spinal cord and up to the brain) to promote neuroplasticity, recovery after stroke or traumatic brain injury, and/or reduce pain.

The signals from the brain in spinal cord injury patients are sometimes blocked or attenuated before reaching the muscles due to the damaged spinal cord. Stimulation over or near the damaged spinal cord pathways raises excitability in those pathways and may facilitate movement and rehabilitation in spinal cord injury patients. Known systems for applying spinal cord stimulation are typically controlled manually through a control pad or device, not by the patient's body motions. According to an embodiment of the disclosure, one or more electrodes 12a, 12b, 12c, . . . 12n are positioned epidurally or preferably transcutaneously over the patient's spinal cord. The system senses particular trajectories made by the patient during physical therapy and, in addition to applying LAMES stimulation to cause muscles to execute a desired motion of a disabled limb, the system triggers transcutaneous spinal cord stimulation, to boost neural signals (by raising excitability of inter-neurons) that have been diminished as a result of spinal cord injury. Likewise, one or more electrodes 12a, 12b, 12c, . . . 12n may be positioned above, over, or below a spinal cord injury site to apply stimulation to the cord injury and/or pathways above and/or below the injury, which may assist healing of neurons impaired by the injury and/or strengthening neuronal connections. By coupling a patient's volitional motion with such stimulation, the patient can control their own stimulation patterns potentially further promoting neuroplasticity and movement and/or sensory function recovery.

For patients who have suffered a stroke, electrical or transmagnetic stimulation over or near the site of the injury in the brain or brain stem may assist in healing of damaged neurons. According to a further embodiment of the disclosure, one or more electrodes on the scalp or a magnetic coil over the scalp are positioned. Stimulation signals are applied to these electrodes or coil in response to a detected motion trajectory, instead of, or preferably in addition to LAMES signals that cause the patient's disabled limb or appendage to move. Such brain stimulation, coupled with the patient's intention to move a disabled limb or appendage, may help restore some of the function of motor neurons injured by the stroke.

Because systems according to the disclosure are relatively inexpensive, portable, and can be controlled by the patient alone, without the help of a therapist or other professional, a patient can be equipped with a device (wearable sleeve(s), patch(es), etc.) they can take home, increasing the hours per week available for rehabilitation.

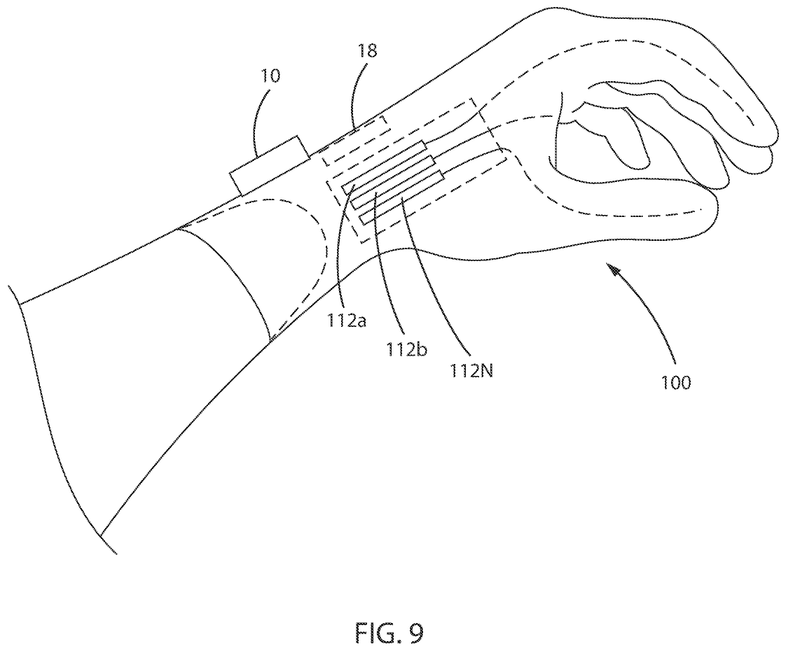

FIG. 9 shows another embodiment of the disclosure. A prosthetic hand 100 is fitted to the arm of a person that has suffered a transradial amputation. The prosthetic hand 100 includes a sensor housing 10. Sensor housing 10 may include sensors 16a, 16b, . . . 16n as discussed above to detect acceleration, velocity, position, and rotation of the wearer's arm. Controller 21 integrating the functions of the MCU 18 and computer 20 discussed in the previous embodiments is connected with the senor array and receives signals indicating motion trajectories executed by the wearer using able-bodied joints, for example, the shoulder, torso, and upper arm. As in the previously described embodiments, controller 21 determines whether the wearer has executed a motion that corresponds with an intended activation of the hand. Controller 21 is connected with actuators 112a, 112b, . . . 112n. These actuators drive motions of the fingers or the prosthesis 100. As with previous embodiments, one or more predetermined trajectories are associated with particular motions of the hand. For example, when the wearer moves his or her upper arm, shoulder, and torso to move the prosthesis in a “rainbow arc,” which as discussed above might indicate the intention to perform a pinch grasp, actuators 112a, 112b, . . . 112n are energized to move the fingers to execute the intended grasping motion.

The embodiment shown in FIG. 9 is for a prosthetic hand 100. The disclosure is not limited to a hand prosthesis. Other types of prostheses can be controlled using a device according to the disclosure. For example, a foot prosthesis could be provided that senses the walking motion of a wearer's leg and operates actuators to orient the foot in synchrony with the wearer's gait.

According to another embodiment, systems according to the disclosure can assist in the training or physical therapy of otherwise able-bodied persons to provide active resistance during exercise. Motion/trajectory recognition of various limbs is used to stimulate non-paralyzed muscles for sports training or physical therapy. For example, the rotation velocity and linear acceleration of a person's forearm is detected using IMU and/or gyroscopic data from sensors mounted on the forearm as part of a sleeve, patch, or other attachment. This motion is normally caused by the bicep. In response to the detected motion, the system triggers antagonist muscles including the triceps to provide active resistance to the bicep, proportional to the forearm's measured rotational velocity. According to one embodiment, the proportionality factor is a settable parameter that allows the user to vary the resistance.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

We claim:

1. A device comprising:
one or more motion sensors, the sensors generating one or more respective motion signals indicative of movement of a first body part of a human;
a muscle stimulator, wherein the muscle stimulator generates one or more stimulation signals to cause one or more muscles to displace a second body part to perform at least one action; and
a processor connected with the one or more motion sensors and the muscle stimulator, the processor including data storage, the data storage including at least one expected trajectory associated with an intention of the human to perform the at least one action, wherein the processor:
receives the one or more signals from the one or more motion sensors;
calculates an actual trajectory of the first body part;
compares the actual trajectory with the expected trajectory; and, based on the comparison,
actuates the muscle stimulator to displace the second body part to perform the at least one action.

2. The device of claim 1, wherein the processor computes a difference between the actual trajectory and the expected trajectory and actuates the muscle stimulator based on the difference.

3. The device of claim 1, wherein the at least one action comprises a plurality of actions, wherein the at least one expected trajectory comprises a plurality of expected trajectories, wherein each of the plurality of expected trajectories is associated with at least one of the plurality of actions, wherein the processor compares the actual trajectory with the plurality of expected trajectories to identify a first trajectory associated with a first action of the plurality of actions, and wherein the processor actuates the muscle stimulator to perform the first action.

4. The device of claim 1, further comprising an input device connected with the processor, the input device adapted to a receive a feedback signal, the feedback signal indicating that the action was the intended action of the human.

5. The device of claim 1, wherein the processor generates the expected trajectory based on a training set of motions.

6. The device of claim 5, wherein the one or more stimulation signals to perform the at least one action comprise a pattern of stimulation signals, and wherein the pattern of stimulation signals is determined from muscle displacements sensed during the training set of motions.

7. The device of claim 6, wherein the muscle displacements are sensed using one or more of an electromyogram sensor, a camera, an inertial motion unit, a bend/joint angle sensor, and a force sensor.

8. The device of claim 1, wherein the processor performs the comparison using one or more of a support vector machine (SVM) algorithm, a hand-writing recognition algorithm, a dynamic time warping algorithm, a deep learning algorithm, a recursive neural network, a shallow neural network, convolutional neural network, a convergent neural network, or a deep neural network.

9. The device of claim 7, wherein the processor performs the comparison using a Long Short-Term Memory type recursive neural network.

10. The device of claim 5, wherein the training set of motions are performed by a second human.

11. The device of claim 5, wherein the training set of motions are performed by the human using a laterally opposite body part of the first body part.

12. The device of claim 1, wherein the motion sensor is located on an arm of the human and wherein the muscle stimulator is adapted to stimulate muscles to move one or more fingers of a hand of the human to perform a grasping motion.

13. The device of claim 1, wherein the expected trajectory is in the shape of an alphanumeric character.

14. The device of claim 12, further comprising an orientation sensor connected with the processor and adapted to monitor an orientation of the first body part, wherein a force applied by the grasping motion depends on an amplitude of the stimulation signal, and wherein the processor adjusts an amplitude of the stimulation signal based, at least in part, on an output of the orientation sensor.

15. The device of claim 14, wherein the processor adjusts the grasping motion to be a key grip, a cylindrical grasp, or a vertical pinch in response to the output of the orientation sensor.

16. The device of claim 12, further comprising a camera connected with the processor and positioned proximate to the hand to capture an image of an object to be grasped, wherein the processor adjusts the grasping motion based in part on the image.

17. The device of claim 12, wherein the processor further comprises a close delay timer, wherein the processor delays stimulating the grasping motion for a predetermined period at the end of the actual trajectory determined by the close delay timer.

18. The device according to claim 12, wherein the processor causes stimulation of the hand to perform a post-grasp activity in response to a post-grasp signal from the motion sensor.

19. The device of claim 18, wherein the post-grasp activity is opening the hand to release the grasp.

20. The device of claim 18, wherein the post-grasp signal is one or more taps of a grasped object against a surface.

21. A device comprising:
one or more motion sensors, the sensors generating one or more respective motion signals indicative of motion of a first body part of a human;
a muscle stimulator, the stimulator generating a stimulation signal adapted to cause or to increase a contraction of a first muscle, wherein the first muscle is a neurologically injured muscle, a paralyzed muscle, a partially paralyzed muscle, or a healthy muscle; and
a processor connected with the sensor and the muscle stimulator, the processor including data storage, the data storage including at least one expected trajectory associated with an intention of the human contract the first muscle, wherein the processor:
receives the one or more motion signals from the one or more sensors;
calculates an actual trajectory of the first body part;
compares the actual trajectory with the expected trajectory;
determines the intention to contract the first muscle based on the comparison; and
causes the stimulator to do one or more of:
cause the contraction of the first muscle;
assist the contraction of the first muscle; and
cause an antagonist contraction of a second muscle, wherein
contraction of the second muscle opposes a movement caused by the contraction of the first muscle.

22. The device of claim 21, further comprising a nerve stimulator connected with, and operable by the processor, wherein, in response to the processor determining the intention to contract the first muscle, the nerve stimulator applies a nerve stimulation signal to a nerve of the human.

23. The device of claim 22, wherein the nerve of the human is selected from one or more of a vagus nerve, a trigeminal nerve, a cranial nerve, a peripheral nerve feeding the first muscle, and a spinal cord of the human.

24. The device of claim 23, wherein the nerve is the spinal cord and wherein the nerve stimulator comprises a transcutaneous electrode positioned above, over, or below a spinal cord injury of the human.

25. A device comprising:
one or more motion sensors, the motion sensors generating one or more respective motion signals indicative of motion of a first body part of a human;
a prosthetic appendage comprising an actuator adapted to change a configuration of the prosthetic appendage to perform an action; and
a processor connected with the one or more motion sensors and the actuator, the processor including data storage, the data storage including at least one expected trajectory associated with an intention of the human to perform the action, wherein the processor:
receives the one or more motion signals from the one or more motion sensors;
calculates an actual trajectory of the first body part;
compares the actual trajectory with the expected trajectory and, based on the comparison,
actuates the actuator to change the configuration of the prosthetic appendage to perform the action.

26. The device of claim 25, wherein the prosthetic appendage comprises a prosthetic hand and wherein the actuator comprises one or more of a wrist actuator and a finger actuator.
